(19) United States
(12) Patent Application Publication
(10) Pub. No.: US 2007/0026443 A1
(43) Pub. Date: Feb. 1, 2007

(54) Diagnosis of uniparental disomy with the aid of single nucleotide polymorphisms

(76) Inventors: Michael Bonin, Rottenburg (DE); Oezge Altug-Teber, Moeglingen (DE); Olaf Riess, Tuebingen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 11/493,863

(22) Filed: Jul. 25, 2006

Related U.S. Application Data
(63) Continuation of application No. PCT/EP05/00855, filed on Jan. 28, 2005.

(30) Foreign Application Priority Data
Jan. 30, 2004 (DE) 10 2004 005 497.5

(51) Int. Cl. C12Q 1/68

(52) U.S. Cl. 435/6

(57) ABSTRACT

The present invention relates to a method for diagnosing uniparental disomy (UPD) in a human being via the analysis of the inheritance of informative single nucleotide polymorphisms (SNPs).

### CROSS-REFERENCES TO RELATED APPLICATIONS

This application is a continuation of copending International Patent Application PCT/EP2005/000855 filed on Jan. 28, 2005 and designating the United States, which was not published under PCT Article 21(2) in English, and claims priority of German Patent Application DE 10 2004 005 497.5, filed on Jan. 30, 2004, which is incorporated herein by reference.

### BACKGROUND OF THE INVENTION

1. Field of the Invention

The present invention relates to a method for diagnosing uniparental disomy (UPD) in a human being.

2. Related Prior Art

Methods of the above type are generally known in the art.

Uniparental disomy (UPD) means the genetic phenomenon in which, contrary to the natural situation, both homologous chromosomes or corresponding chromosome fragments from only one parent are present in an organism due to the absence of separation of a chromosome pair or fragments of the chromosome pair (quote: “non-disjunction”) in meiosis or mitosis. In a case of UPD, either the two homologous chromosomes (heterodisomy) or two copies of a homologous chromosome (isodisomy) or a mixture of heterodisomic and isodiomic segments is found. Depending on which allele is absent, the term used when the paternal allele is absent is maternal disomy, and when the maternal allele is absent it is paternal disomy.

Clinical consequences of UPD emerge when there is a homozygosity case of autosomal recessive traits, through mosaic trisomies or through the change in the expression of genes which are subject to genomic imprinting. In the epigenetic process of imprinting, certain chromosome segments are specifically marked by the male and female germ line so that, in somatic cells, either only the paternal or the maternal allele of a gene is active. Uniparental disomies may in this case lead to complete loss of function of imprinted genes in the corresponding chromosome segments and thus to certain clinical conditions.

Uniparental disomies are frequently observed in miscarriages. Numerical chromosome aberrations are found in 50% of all cases. The incidence of UPD in these zygotes is presumed to be about 3:10 000; cf. Engel, E. (1980) A new genetic concept: uniparental disomy and its potential effect, isodisomie, Am J Med Genet. 6(2): 137-143. The risk of numerical chromosome aberrations and thus of UPD increases with the age of the mothers. A uniparental disomy relating to chromosome 15 (UPD 15) alone is estimated in 1:2400 live births to mothers over 40 years of age; cf. Lalande, M. (1997), “Parental imprinting and human disease”, Rev. Genet. 30: 173-195.

The possible clinical consequences of a UPD are summarized in Table A below (from Robinson, W. P. (2000), “Mechanisms leading to uniparental disomy and their clinical consequences”, Bioessays 22(5): 452-459).

TABLE A
Possible clinical consequences of a UPD

homozygosity for a recessive mutation,
impairment of the imprinting process,
intrauterine growth retardation with postnatal developmental
retardation and fetal malformations because of cells with a UPD in
the placenta and/or fetal tissue,
fertility problems, cancers and other disorders (if the abnormal
cells persist after birth),
a mild phenotype in mosaics (presence of cells with and without UPD),
age-related complex properties such as, for example, in cancers
because of LOH (loss of heterozygosity) or LOI (loss of imprinting)
at the appropriate site of a chromosome.

A UPD is, as is evident from the preceding table 1, frequently associated with an IUGR or PGR (intrauterine growth retardation, postnatal growth retardation). In particular, IUGR and PGR are present as pronounced clinical signs associated with the UPDs of chromosome 16 and 20; cf. Eggermann, E. et al. (2002), Uniparental disomy, clinical indications for testing in growth retardation, Eur J Pediatr. 161(6): 305-12.

The classical examples of a UPD with a characteristic phenotype are Prader-Willi syndrome, which is characterized by a maternal uniparental disomy of chromosome 15 (PWS; matUPD15), the Angelmann syndrome (AS; patUPD15) and the Silver-Russell syndrome (SRS; matUPD7). In addition, clinical manifestations have been described in association with maternal uniparental disomies for chromosomes 14 (stunted growth with developmental retardation), 6 (transient neonatal diabetis mellitus), 11 (Beckwith-Wiedemann syndrome) and 14 (very pronounced stunted growth with mental retardation). It is assumed that uniparental disomies of other chromosomes also have an influence on growth and development.

A large number of diseases which may arise due to a UPD are known and are summarized in table B below (from Engel, E. and Antonorakis, S. E. (2002), Genomic imprinting and uniparental disomy in medicine, Clinical and Molecular Aspects, Wiley-Liss).

TABLE B
Autosomal recessive genetic disorders resulting from a UPD

| Disorder | Gene | UPD |
|---|---|---|
| Junctional epidermolysis bullosa | LAMB3 | matUPD1 |
| Junctional epidermolysis bullosa | LAMC2 | patUPD1 |
| Chediak-Higashi syndrome | LYST | matUPD1 |
| Pycnodysostosis | CTSK | patUPD1 |
| Congenital pain insensitivity with | TRKA | patUPD1 |
| anhidrosis (CIPA) | | |
| 5-Alpha-reductase 2 deficiency | SRD5A2 | patUPD2 |
| Abetalipoproteinemia | MTP | matUPD4 |
| Spinal muscular atrophy | SMA | patUPD5 |
| Complement C4 deficiency | C4 | patUPD6 |
| Methylmalonic acidemia | MUT | patUPD6 |
| 21-Hydroxylase deficiency | CYP21 | patUPD6 |
| Cystic fibrosis | CFTR | matUPD7 |
| Osteogenesis imperfecta | COL1A2 | matUPD7 |
| Congenital chloride diarrhea | DRA | matUPD7 |
| Cystic fibrosis and immotile cilia sydrome | CFTR | matUPD7 |
| Lipoprotein lipase deficiency | LPL | patUPD8 |
| Cartilage-hair hypoplasia | RMRP | matUPD9 |
| Leigh's syndrome | SURF1 | matUPD9 |
| Beta-thalassemia | HBB | patUPD11 |
| Rod monochromacy | | matUPD14 |
| Bloom's syndrome | BLM | matUPD15 |
| Alpha-thalassemia | HBA | patUPD16 |

Diagnosis of a UPD normally takes place in the art by means of microsatellites as genetic markers. Microsatellites are tandem-like, repeating short DNA motifs consisting of nucleotide motifs of up to six base pairs. They occur distributed in the human genome. Microsatellites are highly polymorphic, i.e. the microsatellites from two unrelated individuals differ with high probability.

Microsatellites are inherited in accordance with Mendel's rules. Investigation of whether a UPD is present has to date been carried out by analyzing the inheritance of microsatellites. The DNA of the person to be investigated and of the parents is therefore required for this purpose. This DNA is investigated in a plurality of polymerase chain reactions (PCR) for a plurality of microsatellite markers. Since the microsatellites of the maternal parent differ from those of the paternal parent, it is possible to establish whether, for example, exclusively paternal microsatellites have been inherited, and therefore a paternal UPD is present, in relation to a particular chromosome.

This known UPD diagnostic method in which the inheritance of microsatellites is analyzed has a large number of disadvantages: for a reliable UPD diagnosis, knowledge is required of a considerable number of microsatellites, i.e. microsatellites and, in addition, their flanking sequences must be known for the segments to be investigated in the genome. This means that many, possibly previously unrecognized, UPDs frequently remain undiscovered because of the lack of knowledge about appropriately localized microsatellites. In addition, this UPD diagnostic method is very time-consuming and labor-intensive (diagnosis of a Prader-Willi syndrome or Angelmann syndrome takes two to four weeks) and is therefore carried out only if there is sufficient suspicion of a UPD.

An overview of the UPD diagnostic method carried out in the art is given by Horsthemke, B. et al. (2001), “Leitlinien für die molekulare und zytogenetische Diagnostik bei Prader-Willi-Syndrom und Angelmann-Syndrom”, Verlag Medizinische Genetik, reprint, 7th edition, 78-80.

### SUMMARY OF THE INVENTION

It is therefore an object of the present invention to provide a method for diagnosing UPD in a human being, with which the disadvantages described above are avoided. It is intended in particular to provide a rapid method which can be carried out with few personnel and at low cost and which provides reliable results and also makes it possible to detect previously unknown UPDs.

This object is achieved by a method for diagnosing UPD in a human being which comprises the following steps: (1) provision of a biological sample of the human being and of biological samples of both parents of the human being; (2) genotyping of single nucleotide polymorphisms (SNPs) of the human being and of both parents using the biological samples; (3) selection of informative SNPs of both parents; (4) comparison of the genotypes of the informative SNPs with the genotype of the corresponding SNPs of the human being, and (5) correlation of a homozygosity of the SNPs of the human being from step (4) with the presence of a UPD, or correlation of a heterozygosity of the SNPs of the human being from step (4) with the non-presence of a UPD.

The object underlying the invention is completely achieved by means of this method.

In the context of the invention, a biological sample means one which comprises genomic DNA of the human being to be investigated, such as, for example, a blood sample, saliva sample, a smear, any other cell-containing sample, etc.

SNP means the genetic phenomenon in which the sequences of two unrelated people differ in a single base pair at about every 1000th site. In other words, an SNP designates a position in the genome at which alternatively two different bases occur with a frequency of more than 1%. SNPs are biallelic, i.e. they occur only in two alternatives (A or B for short). The expression of an SNP in a human organism may assume three different genotypes, namely homozygously A (AA), homozygously B (BB) or heterozygously AB (AB).

In the method, after comparison of the genotype of the corresponding SNPs of both parents, the inheritance of these SNPs in the child, i.e. in the human being to be investigated, is examined. This entails according to the invention selection of informative SNPs in both parents. By this SNPs are meant which are present homozygously diametrically opposite in the parents, for example the father has an AA genotype and the mother has a BB genotype of the corresponding SNPs. A child not affected by a UPD then always shows a heterozygous expression (AB of the corresponding SNP). If, on the other hand, a homozygosity, irrespective of the expression (AA or BB), is found for the corresponding SNP of the child or of the organism to be investigated, this allows conclusions to be made about the presence of a uniparental disomy in this genomic region. In this example, the AA homozygosity of the child would correspond to a paternal UPD and the BB homozygosity would correspond to a maternal UPD.

In contrast to microsatellites, an extremely large number of SNPs are known and are distributed with an average distance of 1000 bp over the entire human genome and can be retrieved from databases. To date, almost 1.8 million SNPs have been found in the human genome. Because of this large number of known SNPs and their distribution throughout the genome, as yet unknown forms of UPD can also be detected via analysis of the inheritance of informative SNPs, i.e. even relating to regions of the genome for which no microsatellites are known. In particular, knowledge of the sequences flanking these SNPs is also unnecessary for this purpose.

SNPs are, because of their biallelic form, distinctly less informative than microsatellites which, because of their length of up to six base pairs, may exhibit several thousand genotypic expressions. It has therefore been assumed in the art to date that SNPs are unsuitable as genetic markers for detecting a UPD.

The methods which can be used according to the invention for genotyping the SNPs are all those by means of which it is possible to analyze SNPs preferably over relatively large regions of the genome. Examples thereof are the GeneChip® mapping 10K array supplied by Affymetrix®, with which 11 555 SNPs can be measured in parallel, and further arrays supplied by Affymetrix®, or the BeadArray technology supplied by Illumina with the possibility of measuring 4600 SNPs in parallel.

Compared with the analysis of the inheritance of microsatellites, the method of the invention can be carried out quickly, with few staff and relatively low costs. An additional factor is that small amounts of DNA of the organism to be investigated, namely in the nanogram range, are sufficient for a reliable diagnosis of UPD.

As the inventors have been able to show, a misdiagnosis can be substantially avoided because of the accuracy of the novel method.

It is preferred according to the invention for the genotyping of SNPs of one chromosome to take place in step (2) of the method.

This measure ensures that an adequate number of SNPs is analyzed, so that a reliable diagnosis of UPD is made possible. The risk of a faulty diagnosis is thus reduced.

It is further preferred according to the invention if in step (2) of the method a genome-wide genotyping of SNPs is performed, which further preferably is performed using the GeneChip® mapping 10K array, 100K array or 10K array 2.0 from Affymetrix®.

All the methods have proved to be particularly suitable for use in the context of the method of the invention. Thus, it is possible with the GeneChip® mapping 100K array, which consists of a set of two arrays, to genotype more than 100 000 SNPs with a single primer. The GeneChip® mapping 10K array 2.0 uses photolithography technology, making a smaller and more cost-effective format possible.

As mentioned, the analysis of the inheritance of microsatellites which is known in the art requires information of sequences going beyond the regions to be investigated. This is not the case with the method of the invention, so that it is possible in this embodiment to find previously unknown uniparental disomies genome-wide and also in regions in which no microsatellites are known. Thus, the causes of short stature or symptoms of tall stature and developmental retardation in children are as yet frequently unknown, and it is suspected that many of the children affected have a causative UPD. The present invention now offers the opportunity for the first time to carry out a genome-wide genotyping of the SNPs in these children and to ascertain diagnostically whether a UPD is present in fact or not.

The amounts of DNA required in this embodiment of the invention are only about 250 nanograms, whereas very large amounts in the microgram range would be necessary on application of previous methods in a genome-wide analysis for UPD, and a corresponding investigation would moreover take several months. In contrast thereto, diagnosis of UPD on the basis of a genome-wide genotyping of SNPs by means of the invention is possible within three days. The method of the invention is therefore also suitable as routine investigation.

The high-density distribution of SNPs—the average distance between two SNPs is 210 kB—improves the possibility in particular of detecting small segmental UPDs by comparison with microsatellite analysis—the average distance of microsatellites in the standard set (about 400 microsatellites; cf., for example, www.licor.com) is 5.6 MB (10 cm).

The use of the GeneChip® mapping 10K array from Affymetrix® has the advantage that it is easy to handle because it is designed as microarray, genome-wide analysis of more than 10 000 SNPs is possible, very small initial amounts of DNA are required, and a very high sensitivity and thus reliability is ensured. Further details on this method can be found in the handbook (Manual, 2003 edition), the contents of which are incorporated in the present description by reference.

It is preferred in this connection for the method of the invention to be carried out on a human organism showing signs of a postnatal developmental retardation.

Intrauterine growth retardation (IUGR) with postnatal developmental retardation (PGR) are known to be frequently, but not always, associated with a UPD, but a diagnosis has frequently been avoided even for patients affected because of the high costs and large staff requirement. It is now possible for the first time to make a reliable diagnosis for these people, also routinely and with low cost, by means of the method of the invention to find whether a UPD is present in fact or not, and thus create a basis for a subsequent targeted therapy.

It is particularly preferred in this connection for the method to be carried out on a human organism showing signs of Prader-Willi (PWS), Angelmann (AS), Silver-Russell (SRS) or Beckwith-Wiedemann syndrome or signs of transient neonatal diabetis mellitus.

This measure has the advantage that it is now possible to establish when there are signs of the presence of these syndromes or clinical conditions classically associated with a UPD whether in fact any corresponding genetic situation is present. A diagnostic investigation of people affected has to date been possible only by means of microsatellite analysis, but this has frequently been omitted to date because of the great cost.

With this background, the inventors propose for the first time to use SNPs present in a biological sample of a human organism for diagnosing UPD, in particular in connection with suspected IUGR/PGR, PWS, AS, SRS, Beckwith-Wiedemann syndrome or transient neonatal diabetes mellitus.

It will be appreciated that the features mentioned above and yet to be explained hereinafter can be used not only in the combination indicated in each case, but also in other combinations or alone, without leaving the scope of the present invention.

The present invention is now explained in more detail by means of an exemplary embodiment from which further properties and advantages of the present invention are evident. Reference is made herein to the appended figures.

### BRIEF DESCRIPTION OF THE DRAWINGS

FIG. 1 shows the diagrammatic representation of biallelic SNPs with chromosomal localizations in six unrelated families (Family_{—}1-Family_{—}6). Each informative SNP (marker) is represented by a bar on the right-hand side of the ideogram. The diagnosis in each case is indicated underneath the ideograms (mat: maternal, pat: paternal). The informative markers are distributed in three cases with complete UPD over the entire chromosome (top, families 1 to 3) and in three cases with segmental UPD (bottom, families 4 to 6) over one chromosomal region. In all the cases with the exception of the maternal UPD-15 case (family 2), the SNPs lying between the informative markers showed a reduction to homozygosity, indicating an isodisomy.

FIG. 2 shows the haplotype analysis in the case of the maternal UPD 15 along chromosome 15 (family 2). The reference SNP identity number and the respective genotypes of the selected markers are represented underneath the pedigree (AA: homozygote for one allele, BB: homozygote for the other allele, AB: heterozygote). The chromosomal localizations of these SNPs are listed in the column depicted on the extreme right. The markers at 15q14 to q21.21 and at 15q26.2 to qter showed a reduction to homozygosity, indicating segmental isodisomy (in the square boxes). The informative markers between the heterozygous SNPs along the complete chromosome 15 show an error in meiosis.

FIG. 3 shows a haplotype analysis of the paternal UPD20q case with a group of markers (family 6). The alleles at 20q12 to qter (in the square boxes) show a paternal isodisomy with absence of the maternal distribution in the affected offspring. The SNPs in the closest vicinity to the centromere were not informative and those at 20p showed normal Mendelian inheritance.

### DESCRIPTION OF PREFERRED EMBODIMENTS

Patients Investigated

The method according to the invention was tested on six patients who had previously been diagnosed by means of the conventional diagnostic method based on microsatellite analysis as being affected by Angelmann syndrome (child from family 1; patUPD15), by Prader-Willi syndrome (child from family 2; matUPD15), as being affected by Silver-Russell syndrome (child from family 3; matUPD7), by recurrent abortion, i.e. repeated miscarriage (child from family 4; patUPD2p), by Beckwith-Wiedemann syndrome (child from family 5; patUPD11p) and as being affected by pseudo-hypothyriodism (child from family 6; patUPD20). There was additionally investigation for the respective patients with complete UPD of one parent, families 1, 2 and 3), and with segmental UPD (families 4, 5 and 6) of both parents.

Genotyping of the SNPs of the Parents and Patients, Selection of Informative SNPs

Using the GeneChip® mapping 10K array from Affymetrix® (Affymetrix, Inc., Santa Clara, United States of America), the respective parents and the patients underwent genome-wide analysis for the genotypes of the SNPs in accordance with the manufacturer's instructions (manual). For this purpose, 250 ng of the total genomic DNA was digested with a restriction enzyme (XbaI) and ligated to adapters which recognize the cohesive four base pair-long overhangs. A generic primer which recognizes the adapter sequence was used in order to amplify in a single PCR step the DNA fragments ligated to the adapter. The amplified and purified PCR products were then fragmented and labeled with biotin-ddNTP. The DNA fragments of each sample were then hybridized to a single 10K array in an Affymetrix GeneChip hybridization oven at 48° C. After 16 hours, the arrays were washed and stained in the Affymetrix 400 Fluidics Station. The arrays were scanned with the GeneArray scanner 2500 for families 1, 2 and 3 and with the GeneChip scanner 3000 (Affymetrix, Inc.) for families 4, 5 and 6.

Subsequently, the informative SNPs were selected, i.e. the SNPs which are present homozygously opposite in the parents. Based on statistical considerations, the number of informative SNPs in a genome-wide analysis should be 25 to 100 per family. Subsequently, the SNPs corresponding to the informative SNPs of the parents were selected and their expression was established.

For this purpose, all arrays were analyzed with the Affymetrix GeneChip DNA analysis software 2.0 (GDAS 2.0) in order to generate genotype determinations for each SNP on the array. The genotype determinations were either unambiguous determinations such as homozygous AA, BB, heterozygous AB or no signal. The annotation for each SNP was provided both in GDAS 2.0 and the web-based NetAffxTM analysis center.

The genotype data were then exported into Microsoft Excel (Microsoft Corp., Washington, United States of America) and sorted first according to the chromosomal localization of each SNP and subsequently according to the genotype of the family members. A UPD search over the entire genome took place by identifying biallelic markers which were present homozygously in the patient for one allele and homozygously in the nontransmitting parent for the other allele. Clustered SNPs showing opposite homozygosity were identified as informative biallelic markers indicating the presence of a UPD and were categorized as primary informative markers. All the other SNPs along a chromosome or in a chromosomal segment which were flanked by primary informative markers were evaluated for differentiation between isodisomy and heterodisomy. These SNPs were classified as secondary informative markers. Nonclustered individual SNPs in any chromosomal region showing no Mendelian inheritance were regarded as falsely informative markers.

Result of Diagnosis

The results of the SNP genotyping using the 10K array are summarized in Table 1.

TABLE 1
Summary of the results of the genotyping in families with an offspring affected by uniparental disomy (UPD)

| | | | SNP | | | | |
|---|---|---|---|---|---|---|---|
| | | | determination | Number of | Localization | Number | |
| Family | UPD | Individual | rate | Mi^{a} | of Mi | of Mf^{b} | Chromosomes for Mf^{c} |
| 1 | Paternal | Patient; Angelm. | 85.06% | 47 | 15q11.2-q26.3 | 9 | 1, 2, 5, 11(2), 12(3), 14 |
| | UPD15 | syndrome | | | | | |
| | | Mother | 88.83% | | | | |
| 2 | Maternal | Patient; Prader-Willi | 95.44% | 33 | 15q12-q26.3 | 6 | 1(2), 4, 5, 6, 18 |
| | UPD15 | syndrome | | | | | |
| | | Father | 95.11% | | | | |
| 3 | Maternal | Patient; Silver-Russel | 93.28% | 84 | 7p22.2-q36.3 | 6 | 5, 8(2), 11, 14, 19 |
| | UPD7^{d} | syndrome | | | | | |
| | | Father | 95.41% | | | | |
| 4 | Paternal | Patient; habitual | 87.98% | 65 | 2p25.3-p11.2 | 2 | 9, 16 |
| | UPD2p | abortions | | | | | |
| | Maternal | Mother | 85.14% | 104 | 2q11.2-q37.2 | 4 | 4, 9, 11, 12 |
| | UPD2q^{e} | | | | | | |
| | | Father | 91.43% | | | | |
| 5 | Paternal | Patient; Beckwith- | 85.62% | 31 | 11p15.4-p11.2 | 16 | 2(3), 4(2), 5, 6, 7, 9(2), 11(2), |
| | UPD11p^{↑} | Wiedemann syndrome | | | | | |
| | | Mother | 92.13% | | | | 14, 16, 18(2) |
| | | Father | 91.84% | | | | |
| 6 | Paternal | Patient; pseudo- | 88.31% | 13 | 20q12-q13.33 | 14 | 1, 2, 3, 5(4), 6, 7, 8, 9, 10, 11, 12 |
| | UPD20q^{g} | hypothyriodism | | | | | |
| | | Mother | 84.46% | | | | |
| | | Father | 92.74% | | | | |

^{a}Mi = informative biallelic markers,
^{b}Mf = falsely informative biallelic markers which show no Mendelian inheritance,
^{c}falsely informative markers were localized on various chromosomes listed here. If >1 Mf is found on any single chromosome, the number of these Mf is shown in parentheses,
^{d}Mergenthaler et al., 2000, Formation of uniparental disomy 7 delineated from new cases and a UPD7 case after trisomy 7 rescue. Presentation of own results and review of the literature. Ann Genet 43: 15-21,
^{e}Albrecht et al., 2001; Uniparental isodisomy for paternal 2p and maternal 2q in a phenotypically normal female with two isochromosomes, i(2p) and i(2q). J Med Genet 38: 214,
^{f}Borck et al., 2004, Genome-wide screening using automated fluorescent genotyping to detect cryptic cytogenetic abnormalities in children with idiopathic syndromic mental retardation. Clin Genet 66: 122-127,
^{g}Bastepe et al., 2001, Paternal Uniparental Isodisomy of Chromosome 20q - and the Resulting Changes in GNAS1 Methylation - as a Plausible Cause of Pseudohypoparathyroidism. Am J Hum Genet 68: 1283-1289.

The method carried out would be suitable for reliable UPD diagnosis if the corresponding SNPs were present in homozygous expression in all the patients investigated, and therefore the UPD diagnosis carried out according to the prior art would be confirmed.

The average SNP determination rate in all the families was 90.2% and ranged from 84.46% to 95.44%. These relatively low determination rates compared with the expected average of 95% (Kennedy et al., 2003, Large-scale genotyping of complex DNA. Nat Biotechnol 21:1233-1237) is possibly attributable to the different age of the samples (old samples, repeated freezing and thawing) and the variations caused by the experimenters (Middleton et al., 2004, Genomewide Linkage Analysis of Bipolar Disorder by Use of a High-Density Single-Nucleotide-Polymorphism (SNP) Genotyping Assay: A Comparison with Microsatellite Marker Assays and Finding of Significant Linkage to Chromosome 6q22. Am J Hum Genet 74: 886-897; Shrimpton et al., 2004, A HOX Gene Mutation in a Family with Isolated Congenital Vertical Talus and Charcot-Marie-Tooth Disease. Am J Hum Genet 74:886-897).

In order to estimate the accuracy of the assay for detecting a UPD, all the genotype determinations on the array were investigated for fault determination rates showing opposite homozygosity in the patient and parent (non-Mendelian inheritance). Only 0.03 to 0.1% of the SNPs showed such false-positive determinations, which have been defined in this study also as false-positive markers (table 1). In isodisomic chromosomal regions in all the analyzed patients there were only three false-negative determinations (0.2%). However, these SNPs were localized on different chromosomes and were not clustered in a chromosomal region. In addition, the X chromosome genotype determinations were analyzed in eight male individuals. In this case, on the basis of a single X chromosome, a homozygosity was expected for each SNP on the X chromosome. None of the SNPs mapped on the X chromosome showed heterozygosity in the eight male individuals investigated (data not shown).

All the selected SNPs distributed over the entire genome of the patients investigated corresponding to the informative SNPs of the parents were present in the homozygous state and thus verify the UPD diagnosis undertaken previously.

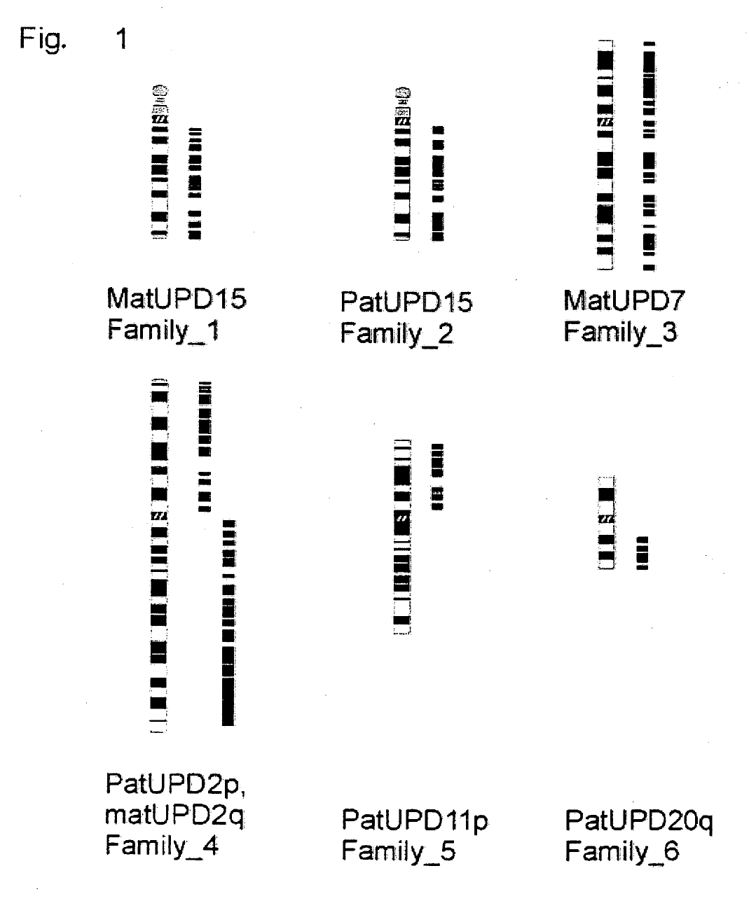

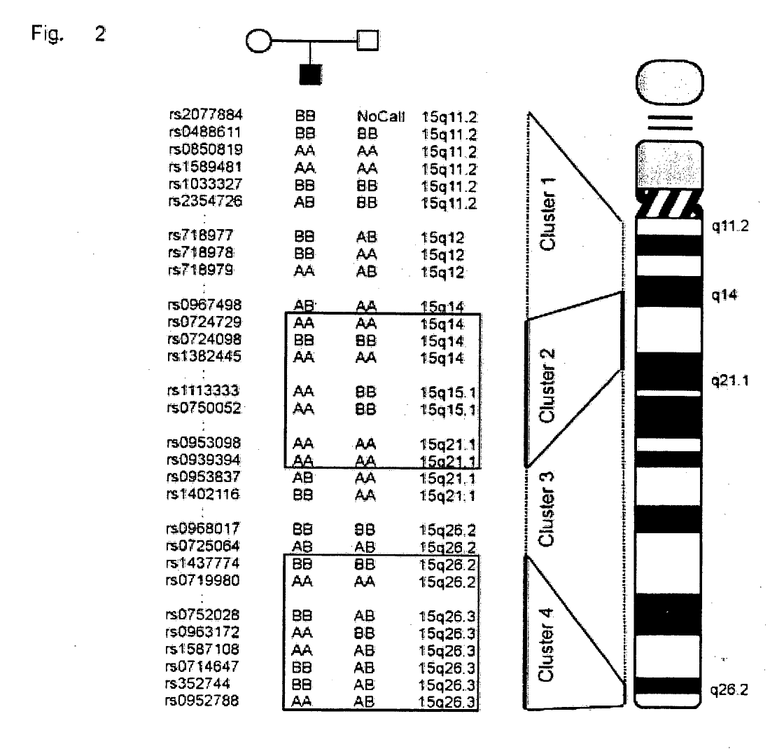

In family 2 (maternal UPD 15), 33 informative markers were identified on chromosome 15 (Table 1, FIG. 1). The genotyping analysis of this patient revealed that heterozygous and homozygous determination were distributed randomly over the chromosome, with the exception of two clusters of homozygous determinations at 15 q14 to q21.1 (cluster 2) and at 15 q26.2 to qter (cluster 4) (FIG. 2). The patient showed only a single heterozygous SNP within cluster 2 (false-negative determination). The first five SNPs in the closest vicinity to the centromere at 15 q1.2 were identically homozygous in the patient and the father, followed by a cluster of heterozygous SNPs (cluster 1) (FIG. 2). The presence of heterozygous markers along the entire chromosome 15 correlates with an error in meiosis.

In the case of the paternal UPD 15 (family 1), 47 informative biallelic markers were detected on chromosome 15, whereas in the case of the maternal UPD 7 (family 3), 84 informative markers were identified on chromosome 7 (Table 1, FIG. 1). In each case there was only one heterozygous determination in the 297 detected SNPs on chromosome 15 and in the 559 detected SNPs on chromosome 7 (false-negative determinations). This reduction to homozygosity over the entire genome in both cases indicates the complete isomy of the corresponding chromosomes, which might be attributable to a postzygotic error.

In order to investigate the efficiency of the microarray-based method of the invention in relation to the identification of segmental UPDs, the 10K array was investigated in three different cases, one paternal UPD 20q (pseudo-hypothyriodism), one paternal UPD11p (Beckwith-Wiedemann syndrome) and one paternal UPD2p in combination with a maternal UPD2q (recurrent abortion). The latter patient (family 4) had a rare chromosomal rearrangement with two isochromosomes, i(2p) and i(2q); cf. Albrecht et al., 2001 (loc. cit.). The two different UPDs present in this patient were evaluated separately. It was possible to detect 65 and 104 informative markers respectively on 2p and 2q (Table 1, FIG. 1). The reduction to homozygosity of all the other SNPs on 2p and on 2q confirmed paternal uniparental isodisomy 2p and maternal uniparental isodisomy 2q.

In family 5 (paternal UPD 11p) (Borck et al., 2004, loc. cit.), 31 informative markers were localized between 11p15.4 and 11p11.2 (FIG. 1) with reduction to homozygosity of all biallelic markers, indicating isodisomy in this region. The SNPs in the vicinity of the centromeres showed either a normal biallelic inheritance or were noninformative.

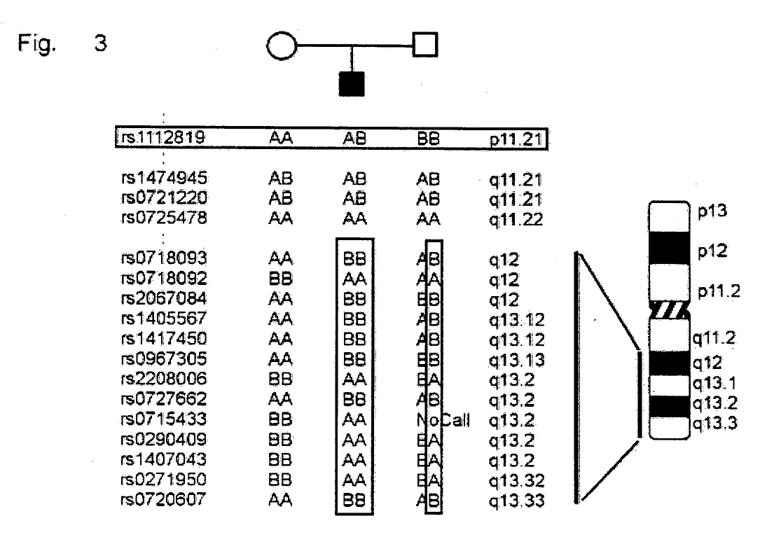

In family 6 (paternal UPD20q) (Bastepe et al., 2001, loc. cit.), in total 13 informative markers were identified over the long arm of chromosome 20 (Table 1, FIG. 1). All 91 detected SNPs, including the informative markers between 20q11.22 to q13.33, showed a reduction to homozygosity corresponding to an isodisomy of this segment. At the end, only two SNPs in the closest vicinity to the centromere (in 20q 11.21) showed heterozygosity in the patient and the parents (FIG. 3). The SNPs localized in 20p showed normal Mendelian inheritance (FIG. 3).

Further control experiments were carried out in parallel therewith. In these, healthy subjects and their parents were genotyped for informative SNPs. It emerged that the corresponding SNPs of the healthy subjects were present exclusively in the heterozygous state.

These results demonstrate that the presence of a homozygous expression in the investigated patient of the SNPs which correspond to the informative SNPs of the parents correlates with the presence of a UPD. It is therefore possible with the method of the invention to diagnose uniparental disomy reliably on the basis of the genome-wide analysis of the inheritance of informative SNPs.

Therefore, what is claimed, is:

1. A method for diagnosing uniparental disomy (UPD) in a human being which comprises the following steps:
(1) providing a biological sample of the human being and of biological samples of both parents of the human being;
(2) genotyping of single nucleotide polymorphisms (SNPs) of the human being and of both parents using the biological samples;
(3) selecting informative SNPs of both parents;
(4) comparing the genotypes of the informative SNPs with the genotype of the corresponding SNPs of the human being, and
(5) correlating a homozygosity of the SNPs of the human being from step (4) with the presence of a UPD, or correlation of a heterozygosity of the SNPs of the human being from step (4) with the non-presence of a UPD.

2. The method of claim 1, wherein in step (2) the genotyping of SNPs of one chromosome is performed.

3. The method of claim 1, wherein in step (2) a genome-wide genotyping of SNPs is performed.

4. The method of claim 3, wherein the genome-wide genotyping in performed by means of a method supplied by Affymetrix® which is selected from the group consisting of: GeneChip® mapping 10K array, GeneChip® mapping 100K array, GeneChip® mapping 10K array 2.0.

5. The method of claim 1, wherein the human being shows signs of postnatal developmental retardation (IUGR/PGR).

6. The method of claim 1, wherein the human being shows signs of a syndrome which is selected from the group consisting of: Prader-Willi syndrome (PWS), Angelmann syndrome (AS), Silver-Russell syndrome (SRS) and Beckwith-Wiedemann syndrome.

7. The method of claim 1, wherein the human being shows signs of transient neonatal diabetes mellitus.
